(19) United States
(12) Patent Application Publication
(10) Pub. No.: US 2003/0027760 A1
(43) Pub. Date: Feb. 6, 2003

(54) Composition and methods to improve neural outcome

(76) Inventors: Peter David Gluckman, Auckland (NZ); Ernest S. Sirimanne, Auckland (NZ); Geoffrey W. Krissansen, Auckland (NZ); Jagat R. Kanwar, Auckland (NZ)

Correspondence Address: HELLER EHRMAN WHITE & MCAULIFFE LLP, 275 MIDDLEFIELD ROAD, MENLO PARK, CA, 94025-3506, US

(21) Appl. No.: 10/119,386

(22) Filed: Apr. 8, 2002

Related U.S. Application Data
(63) Continuation-in-part of application No. 09/910,461, filed on Jul. 20, 2001.
Application No. 09/910,461 is a continuation of application No. 08/907,918, filed on Aug. 11, 1997, now abandoned.
Application No. 08/907,918 is a continuation of application No. 08/656,331, filed on Jun. 14, 1996, now abandoned.
Application No. 08/656,331 is a 371 of international application No. PCT/NZ94/00143, filed on Dec. 20, 1994.

(30) Foreign Application Priority Data
Dec. 23, 1993 (NZ) 250572
Mar. 14, 1994 (NZ) 260091
Jul. 22, 1994 (NZ) 264070

(51) Int. Cl. A61K 38/18

(52) U.S. Cl. 514/12

(57) ABSTRACT

A method for protecting white matter, axons, and oligodendrocytes of a mammal, especially a human, against degeneration and death resulting from multiple sclerosis or periventricular leucomalacia by increasing the effective concentration of a GPE-related compound in the central nervous system of the mammal. This increase may be achieved by administration to the mammal of an effective amount of a GPE-related compound, a prodrug thereof, or an implant containing cells that express the GPE-related compound or prodrug. The use of a GPE-related compound, a prodrug thereof, or an implant containing cells that express the GPE-related compound or prodrug in the manufacture of a medicament for protecting white matter, axons, and oligodendrocytes of a mammal, especially a human, against degeneration and death resulting from multiple sclerosis or periventricular leucomalacia; and compositions containing a GPE-related compound, a prodrug thereof, or an implant containing cells that express the GPE-related compound or prodrug for protecting white matter, axons, and oligodendrocytes of a mammal, especially a human, against degeneration and death resulting from multiple sclerosis or periventricular leucomalacia.

### CROSS-REFERENCE TO RELATED APPLICATIONS

This application is a continuation-in-part of application Ser. No. 09/910,461, filed Jul. 20, 2001; which is a continuation of application Ser. No. 08/907,918, filed Aug. 11, 1997, abandoned; which is a continuation of application Ser. No. 08/656,331, filed Jun. 14, 1996, abandoned; which is a 371 of PCT International Application No. PCT/NZ94/00143; filed Dec. 20, 1994; which claims priority from New Zealand Applications Nos. 250572, filed Dec. 23, 1993; 260091; filed Mar. 14, 1994; and 264070, filed Jul. 22, 1994. These applications are incorporated into this application by reference.

### BACKGROUND OF THE INVENTION

1. Field of the Invention

This invention relates to a method for protecting white matter, axons, and oligodendrocytes of a mammal, especially a human, against degeneration and death resulting from multiple sclerosis or periventricular leucomalacia.

2. Description of the Related Art

The central nervous system is peculiar among mammalian organs in that differentiated neurons are practically incapable of regeneration. Permanent loss of function is a likely outcome of a sufficiently severe injury to the brain. There is therefore a need for means to protect cells of the central nervous system (also including the glial cells) from death after an injury.

After asphyxial, traumatic, toxic, infectious, degenerative, metabolic, ischemic or hypoxic insults to the central nervous system (CNS) of man or other mammals a certain degree of damage in several different cell types may result. For example periventricular leucomalacia, a lesion which affects the periventricular oligodendrocytes is generally considered to be a consequence of hypoxic ischemic injury to the developing preterm brain (Bejar et al., Am. J. Obstet. Gynecol., 159: 357-363 (1988); Sinha et al., Arch. Dis. Child., 65: 1017-1020 (1990); Young et al., Ann. Neurol, 12: 445-448 (1982)). Damage to the CNS by trauma, asphyxia, ischemia, toxins or infection is frequent and may cause sensory, motor or cognitive deficits. Glial cells which are non-neuronal cells in the CNS are necessary for normal CNS function. Infarcts are a principal component of some hypoxic ischemic induced damage and loss of glial cells is an essential component of infarction. There appears to be a kind of “delayed injury process” in which apparently “self destructive” neural activity occurs some time after an injury; attempts to control this activity appear able to alleviate the effects of this delayed injury process.

Disease of the CNS also may cause loss of specific populations of cells. For example multiple sclerosis is associated with loss of myelin and oligodendrocytes. Some situations in which CNS injury or disease can lead to predominant loss of neurons and/or other cell types include: perinatal asphyxia associated with fetal distress such as following abruption, cord occlusion or associated with intrauterine growth retardation; perinatal asphyxia associated with failure of adequate resuscitation or respiration; severe CNS insults associated with near-miss drowning, near miss cot death, carbon monoxide inhalation, ammonia or other gaseous intoxication, cardiac arrest, collapse, coma; meningitis, hypoglycemia and status epilepticus; episodes of cerebral asphyxia associated with coronary bypass surgery; cerebral anoxia or ischemia associated with stroke, hypotensive episodes and hypertensive crises; and cerebral trauma.

There are many other instances in which CNS injury or disease can cause damage to cells of the CNS. It is desirable to treat the injury in these instances. Also, it is desirable to prevent or reduce the amount of CNS damage which may be suffered as a result of induced cerebral asphyxia in situations such as cardiac bypass surgery.

We have previously shown in New Zealand Patent Application No. 239211 that the growth factor called insulin-like growth factor 1 (IGF-1) has an unanticipated action, namely to prevent brain cells from dying after an asphyxial or ischemic brain insult (Gluckman et al., Biochem. Biophys. Res. Commun., 182: 593-599 (1991)). Because insulin also has a neuroprotective action (Voll et al., Neurology, 41:423-428 (1991)), and because insulin and IGF-1 can both bind to the IGF-1 receptor, it was generally assumed that this brain rescue mode of action of IGF-1 was mediated via the IGF-1 receptor (Guan et al., J. Cereb. Blood Flow Metab., 13: 609-616 (1993)).

It is known that IGF-1 can be modified by proteolytic cleavage in nervous tissue to des 1-3N IGF-1, that is IGF-1 missing the 3 amino acids from the amino terminal of the molecule, and hence after cleavage there is also a 3 amino acid peptide gly-pro-glu which is the N terminal tripeptide. This tripeptide is also termed GPE. As des 1-3N IGF-i also binds to the IGF-1 receptor and GPE does not, GPE was thought to be of no significance to the neuronal rescue action of IGF-1. To date, there has been no enabling reference in the prior art to the manipulation of the cleaved tripeptide GPE itself to prevent or treat CNS injury or disease leading to CNS damage in vivo.

One disease which leads to CNS damage in vivo is multiple sclerosis. Multiple sclerosis belongs to a group of neurological disorders known as demyelinating conditions. In demyelinating conditions the lipid protein composite substance myelin, that surrounds the axons of neurons and increases their ability to conduct electrical signals, degenerates. This group of conditions includes acute and chronic encephalomyelitis, optic neuritis, transverse myelitis, Devic's disease, the leucodystrophies, multiple sclerosis, progressive multifocal leukoencephalopathy, central pontine myelinolysis, neuromyelitits optica, diffuse cerebral sclerosis of Schilder, acute and subacute necrotizing haemorrhagic encephalitis.

Multiple sclerosis affects 350,000 Americans and is, with the exception of trauma, the most frequent cause of neurologic disability in early to middle adulthood. Multiple sclerosis is a demyelinating, neurological disorder of which there are generally considered to be 3 forms: relapsing, progressive and inactive. The disease is characterized by selective demyelination of CNS axons, inflammation, and gliosis. Neurons communicate by sending electrical impulses along cellular processes called axons. Axons are insulated with a protein-lipid composite substance called myelin. Myelin is produced by specialized cells (generically referred to as glia). In the central nervous system, the main myelin producing glia are named oligodendrocytes. Injury to myelin sheaths surrounding axons may interrupt communication between neurons and produce myelin breakdown. When myelin sheaths or oligodendrocytes sustain injury, entire segments of myelin degenerate, and their remnants are phagocytosed by macrophages and to a much lesser degree by astrocytes. This is called “primary demyelination” if most axons remain uninjured and is characteristic of the myelin breakdown seen in multiple sclerosis. Multiple sclerosis derives its name from the multiple scarred areas visible on macroscopic examination of the brain. Areas of tissue affected in this manner are called plaques, and range in size between 1 mm and several centimeters. Demyelinating lesions are historical evidence of the occurrence of or the continued presence of perivascular lesions. Occasionally, plaques are also present in gray matter (neuron cell bodies). The multiple sclerosis lesion is defined as including both perivascular and demyelinating lesions. Demyelination along with causing conduction abnormalities between neurons can in severe cases lead to premature death.

Myelin is produced by specialized cells (generically referred to as glia). In the CNS the main myelin producing glia are named oligodendrocytes. Injury to myelin sheaths surrounding axons may interrupt communication between neurons and produce myelin breakdown. When myelin sheaths or oligodendrocytes sustain injury, entire segments of myelin degenerate, and their remnants are phagocytosed by macrophages and to a much lesser degree by astrocytes. This is called “primary demyelination” if most axons remain uninjured and is characteristic of the myelin breakdown seen in multiple sclerosis. In multiple sclerosis, and in experimental autoimmune encephalomyelitis (EAE), an animal model of multiple sclerosis, encephalitogenic leukocytes penetrate the blood brain barrier and damage the myelin sheath of nerve fibres (Raine, Neuropath. Appl. Neurobio., 17: 265-274 (1991); Raine, Ann. Neurol., 36: S61-S62 (1994); Martin et al., Crit. Rev. Clin. Lab. Sci., 32:121-182 (1995); Cannella et al., Ann. Neurol, 37: 424-435 (1995); Brosnan et al., Brain Pathol., 6: 243-257 (1996)).

The cause of multiple sclerosis is unknown but is thought to have an autoimmune etiology initiated by exposure to a virus in genetically predisposed individuals. Current drug management of the disease consists of immunosuppression to arrest the disease process and for arresting the symptoms. No drugs are currently successful in intervening in the disease process.

Each document cited here or elsewhere in this application is incorporated into this application by reference.

### SUMMARY OF THE INVENTION

In a first aspect, this invention is a method for protecting white matter, axons, and oligodendrocytes of a mammal, especially a human, against degeneration and death resulting from multiple sclerosis or periventricular leucomalacia comprising increasing the effective concentration of a GPE-related compound in the central nervous system of the mammal. This increase may be achieved by administration to the mammal of an effective amount of a GPE-related compound, a prodrug thereof, or an implant containing cells that express the GPE-related compound or prodrug.

In another aspect, this invention is the use of a GPE-related compound, a prodrug thereof, or an implant containing cells that express the GPE-related compound or prodrug in the manufacture of a medicament for protecting white matter and axons of a mammal, especially a human, against degeneration and the death of oligodendrocytes resulting from multiple sclerosis or periventricular leucomalacia ; and compositions containing a GPE-related compound, a prodrug thereof, or an implant containing cells that express the GPE-related compound or prodrug for protecting white matter and axons of a mammal, especially a human, against degeneration and the death of oligodendrocytes resulting from multiple sclerosis or periventricular leucomalacia.

### BRIEF DESCRIPTION OF THE DRAWINGS

FIG. 1 shows the clinical severity score in rats following treatment with a control vehicle or with 150 μg GPE per day between days 1-9 after an experimental autoimmune encephalornyelitis (EAE) lesion.

FIG. 2 shows that the combination of the neuroprotectors GPE and NBQX causes sustained remission of advanced EAE, which is further enhanced by anti-MAdCAM-1 antibody blockade. FIG. 2A shows that neuroprotectors and cell adhesion blockade with an anti-MAdCAM-1 antibody attenuate the early development of EAE. FIG. 2B shows that a combination of GPE and NBQX caused sustained remission of advanced EAE, which is further enhanced by anti-MAdCAM-1 antibody blockade. FIG. 2C shows that prolonged combination therapy is non-toxic, and provides sustained protection. Arrows indicate antibody administration, and the speckled line indicates neuroprotector administration.

FIG. 3 shows the changes in average body weight associated with disease progression and remission following therapy. Arrows indicate antibody administration, and the speckled line indicates neuroprotector administration.

FIG. 4 shows apoptosis in the CNS, as assessed by TUNEL staining. Arrows indicate antibody administration, and the speckled line indicates neuroprotector administration.

FIG. 5 shows the assessment of axonal damage by measuring levels of abnormally dephosphorylated neurofilament H. FIG. 5A is a Western blot analysis of dephosphorylated neurofilament H in spinal cord homogenates. FIG. 5B is a graph showing numbers of damaged axonal cells (cells staining for dephosphorylated neurofilament H). FIG. 5C is a densitometric analysis of the Western blots of FIG. 5A.

FIG. 6 shows oligodendrocyte survival from measurement CNPase-immunoreactivity. FIG. 6A is a graph showing numbers of oligodendrocytes in transverse sections of dorsal columns. FIG. 6B is a graph showing oligodendrocyte loss in the CNS of treated versus mock treated and untreated mice. FIG. 6C is a Western blot analysis of CNPase in spinal cord homogenates.

FIG. 7 shows that glutamate receptor subunit upregulation is blocked by therapy. FIG. 7A is a Western blot analysis of GluR2 subunit expression in spinal cord homogenates. FIG. 7B is a Western blot analysis of NMDAR1 subunit expression in spinal cord homogenates.

### DETAILED DESCRIPTION OF THE INVENTION

Definitions

A “GPE-related compound” is GPE (the tripeptide gly-pro-glu) or a GPE analog, and is a neuroprotective agent.

A “GPE analog” is a small peptide (not more than 5 amino acids) or peptidomimetic (a compound where one or more of the amide bonds of such a peptide is replaced by a non-amide bond) that is capable of effective binding to mammalian central nervous system GPE receptors. Preferred GPE analogs are those capable of producing an effect substantially equivalent to that produced by GPE itself. GPE analogs include the dipeptides gly-pro (GP)and pro-glu (PE), GPE amide, GPE steatate, gly-pro-D-glutamate (GP-D-E), gly-pro-thr (GPI), gly-glu-pro (GEP), glu-gly-pro (EGP), glu-pro-gly (EPG), pro-gly-glu (PGE), and pro-glu-gly (PEG).

A “prodrug” of a GPE-related compound is a compound comprising the GPE-related compound and a carrier linked to the GPE-telated compound by chemical bond(s) that are cleaved by biological processes within a mammal when the prodrug is administered to the mammal, such as by the action of enzyme(s) present within the mammal. Prodrugs include, for example, esters of the GPE-related compound, such as the 1-[(ethoxycarbonyl)oxy]ethyl ester, and polypeptides that, when cleaved by a mammalian enzyme, yield the GPE-related compound. Suitable enzymes include an acid protease that generates des-(1-3) IGF-1 and GPE from IGF-1 (Yamamoto et al., Endocrinology, 135(6): 2432-2439 (1994)), proprotein and prohormone convertases (Seidah et al., Brain Research, 848: 45-62 (1999)), serum proteases, trypsin (in a calcium/magnesium-free solution), cathepsin-D, and pepstatin-A.

“Protecting white matter, axons, and oligodendrocytes of a mammal” by increasing the effective concentration of a GPE-related compound in the central nervous system of the mammal includes both the prevention of initial degradation or death of (“damage to”) the white matter, axons, and oligodendrocytes of the mammal resulting from multiple sclerosis of periventricular leucomalacia, the prevention of further damage when one or more of the white matter, axons, or oligodendrocytes have already suffered damage, and the rescue or repair of one or more of white matter, axons, and oligodendrocytes when they have already suffered damage.

An “effective amount” of a GPE-related compound, prodrug, or implant is that amount of such compound, prodrug, or implant that, when administered to a mammal requiring protecting white matter, axons, and oligodendrocytes, produces an increase in effective concentration of a GPE-related compound in the central nervous system of the mammal sufficient to promote protection of white matter, axons, and oligodendrocytes in that mammal.

Description and Preferred Embodiments

In a first aspect, this invention is a method for protecting white matter, axons, and oligodendrocytes of a mammal, especially a human, against degeneration and death resulting from multiple sclerosis or periventricular leucomalacia comprising increasing the effective concentration of a GPE-related compound in the central nervous system of the mammal. This increase may be achieved by administration to the mammal of an effective amount of a GPE-related compound, a prodrug thereof, or an implant containing cells that express the GPE-related compound or prodrug.

In another aspect, this invention is the use of a GPE-related compound, a prodrug thereof, or an implant containing cells that express the GPE-related compound or prodrug in the manufacture of a medicament for protecting white matter and axons of a mammal, especially a human, against degeneration and the death of oligodendrocytes resulting from multiple sclerosis or periventricular leucomalacia ; and compositions containing a GPE-related compound, a prodrug thereof, or an implant containing cells that express the GPE-related compound or prodrug for protecting white matter and axons of a mammal, especially a human, against degeneration and the death of oligodendrocytes resulting from multiple sclerosis or periventricular leucomalacia.

We have explored the observation that insulin-like growth factor 1 (IGF-1) appears to be modified by proteolytic cleavage in nervous tissue to des 1-3N IGF-1, that is IGF-1 missing the three amino acids from the amino terminal of the molecules, and to GPE, which is the N terminal tripeptide. As des 1-3N IGF-1 also binds to the IGF-1 receptor and GPE does not, GPE was thought to be of no significance to the neuronal rescue action of IGF-1. Surprisingly, GPE is effective. To date, there has been no enabling reference in the prior art to the manipulation of GPE to prevent or treat CNS injury or disease leading to CNS damage in vivo.

Surprisingly we have found that GPE itself promotes neural rescue. This has led us to propose that treating a patient for neural injury or disease with IGF-1 is a less soundly based proposition, as a tripeptide is easier to prepare, can cross the blood-brain barrier, and as it is a more mobile and less imamunologically challenging compound therefore it can be expected to be more effective.

EP 366638 has shown GPE to modulate neuronal activity and, because agents such as NMDA which do, may have some role in treating neuronal injury, suggested but did not provide any evidence for its use as a treatment for neurological disease. However there is no prior art for our claims which are that GPE can be used to prevent and/or attenuate neurological disease by preventing neurons and glia from dying. The type of clinical application of which our invention is directed is totally different from EP 366638.

In still a further aspect, the present invention comprises a therapeutic kit for the treatment of multiple sclerosis or periventricular leucomalacia, the kit comprising a GPE-related compound or prodrug and GPE analogues and peptidomimetics or a prodrug thereof.

The preferred GPE-related compound is GPE.

The increase in the effective concentration of a GPE-related compound in the central nervous system of the mammal may be achieved by administration to the mammal of an effective amount of the GPE-related compound, a prodrug thereof, or an implant containing cells that express the GPE-related compound or prodrug. The administration may be either prophylactic (after identification of a mammal with a predisposing or precipitating factor for multiple sclerosis or periventricular leucomalacia, but prior to a diagnosis of the same), therapeutic (after diagnosis of the same has been made), or both. Additionally, administration of a GPB-related compound may be continued to prevent relapse in the previously affected area.

The GPE-related compound or prodrug can be administered alone, or as is preferred, as a part of a pharmaceutical composition or medicament. In general, GPE compounds will be administered as pharmaceutical compositions by one of the following routes: directly to the central nervous system, oral, topical, systemic (e.g. transdermal, intranasal, or by suppository), parenteral (e.g. intramuscular, subcutaneous, or intravenous injection), by implantation and by infusion through such devices as osmotic pumps, transdermal patches and the like. Compositions may take the form of tablets, pills, capsules, semisolids, powders, sustained release formulation, solutions, suspensions, elixirs, aerosols or any other appropriate compositions; and comprise at least GPE-related compound or prodrug in combination with at least one pharmaceutically acceptable excipient. Suitable excipients are well known to persons of ordinary skill in the art, and they, and the methods of formulating the compositions, may be found in such standard references as Gennaro, ed., “Remington: The Science and Practice of Pharmacy”, 20th ed., Lippincott, Williams & Wilkins, Philadelphia Pa. (2000). Suitable liquid carriers, especially for injectable solutions, include water, aqueous saline solution, aqueous dextrose solution, and the like, with isotonic solutions being preferred for intravenous administration.

The GPE or other GPE-related compound, or prodrug can be administered directly to the central nervous system. This route of administration can involve, for example, lateral cerebroventricular injection, focal injection, or a surgically inserted shunt into the lateral cerebral ventricle. In such a case, the GPE-related compound can be administered in conjunction with artificial cerebrospinal fluid.

An advantage of GPE and other GPE-related compounds is that they can be administered peripherally and have both peripheral and central nervous system effects. Thus, GPE and other GPE-related compounds and prodrugs need not be administered directly to the central nervous system in order to have effect in the central nervous system. Any peripheral route known in the art can be employed. Two particularly convenient peripheral administration routes are by subcutaneous injection (e.g. dissolved in 0.9% sodium chloride) and by oral administration (e.g., in a tablet or capsule).

GPE and other GPE-related compounds and prodrugs can also be administered by a sustained-release system. Suitable examples of sustained-release compositions include semi-permeable polymer matrices in the form of shaped articles, e.g. films, or microcapsules. Sustained-release matrices include polylactides (U.S. Pat. No. 3,773,919; EP 58481), copolymers of L-glutamic acid and y-ethyl-L-glutamate (Sidman et al., Biopolymers, 22: 547-556 (1983)), poly(2-hydroxyethylmethacrylate) (anger et al., J. Biomed. Mater. Res., 15: 267-277 (1981)) ethylene vinyl acetate (Langer et al., supra), or poly-D-(-)-3-hydroxy-butyric acid ASP 133988). Sustained-release compositions also include liposomally entrapped compounds. Liposomes containing the compound are prepared by methods known per se: DE 3218121; Hwang et al., Proc. Nat'l Acad. Sci. USA, 77: 4030-4034 (1980); EP 52322; EP 36676; EP 88046; EP 143949; EP 142641; JP 83-118008; U.S. Pat. No. 4,485,045, U.S. Pat. No. 4,544,545; and EP 102324. Ordinarily, the liposomes are of the small (from about 20 to about 80 nm diameter) unilamellar type in which the lipid content is greater than about 30 mole percent cholesterol, the selected proportion being adjusted for the most efficacious therapy. Other sustained-release systems include implantable osmotic systems of the type described in U.S. Pat. No. 5,980,508. The GPE-related compounds and prodrugs may also be PEGylated to increase their lifetime in vivo, based on, e.g. the conjugate technology described in WO 95/32003. Mechanical devices providing sustained infusion, such as those commonly used for the delivery of insulin, may also be used.

The effective concentration of GPE or other GPE-related compounds can also be increased by the use of an implant which is or includes a stable expression cell line which is capable of expressing the GPE-related compound in an active form within the body, or more particularly the central nervous system of the patient (Martínez-Serrano et al., Proc. Nat'l Acad. Sci. USA, 95: 1858-1863 (1998); Chen et al., J. Neurosci., 15(4): 2819-2825 (1995)). Cells such as astrocytes (Yoshimoto et al., Brain Res., 691: 25-36 (1995)), fibroblasts (Chen et al., J. Neurosci., 15(4): 2819-2825 (1995); Frim et al., Proc. Nat'l Acad. Sci. USA, 91: 5104-5108 (1994)), HiB5 cells (Martínez-Serrano et al., Proc. Nat'l Acad. Sci. USA, 95: 1858-1863 (1998)), and baby hamster kidney cells (Tseng et al., J. Neurosci., 17(1): 325-333 (1997)), either primary cells or cell lines, immortalized or not, and engineered to express the GPE-related compound may be implanted into the brain or elsewhere in the body, or encapsulated in biocompatible polymers, fibers or other materials and the cell-containing capsules then implanted into the brain or elsewhere in the body. Cells may be cultured in Dulbecco's Modified Eagle's Medium (DMEM) containing 10% fetal calf serum and 1% penicillin/streptornycin prior to encapsulation and/or implantation. Cells to be encapsulated may be suspended in a solution of 1:1 culture medium:3% collagen at a density of 500,000 cells/μL. This cell suspension can then be encapsulated in capsules such as poly(ether-sulfone)fibers from AKZO-Fiber Nobel AG, Wupperthal, Germany). Capsules are preferably cultured for 4 days before implantation.

Engineering cells to express a GPE-related compound in active form may be achieved through the use of an expression vector. For example, for GPE, multiple copies of any DNA sequence specific for the amino acids methionine/glycine/proline/glutanic acid and a stop codon are linked together, either with or without additional DNA sequences specific for a stop codon between each 4 amino acid sequence, to form a complete sequence of between 30-50 nucleotides. Note that the start codon will always be ATG, the codon for methionine, whereas the stop codon may be either TAA, TAG or TGA. This complete sequence comprises the expression vector for GPE. The expression vector as a whole will generally also include a promoter for the cell to be implanted, and may include selection markers and other DNA sequences common in the biotechnology field. This vector may then be episomally expressed or integrated into the genome of the cells to be implanted.

The calculation of the effective amount of GPE-related compound or prodrug to be administered will be dependent upon the route of administration and upon the nature of the condition which is to be treated, and will be routine to a persons of ordinary skill in the art. For a human, where the dose is administered centrally, a suitable dose range for GPE is between about 0.1 μg and about 400 μg per Kg of body weight per day; a preferred dose range is between about 0.5 μg/Kg/day and about 50 μg/Kg/day, and a more preferred dose range is from about 1 μg/Kg/day to about 25 μg/Kg/day. For peripheral administration, the doses are about 10-fold to 1000-fold higher; and suitable dose ranges will be readily determinable by comparing the activities of peripherally administered GPE with the activity of centrally-administered GPE in a suitable model and scaling the central GPE dose range above accordingly. Suitable dose ranges for other GPE-related compounds will be readily determinable by comparing the activities of the compounds with the activity of GPE in a suitable model and scaling the GPE dose range above accordingly; and suitable dose ranges for prodrugs and implants will be determinable in the same manner.

The GPE-related compound or prodrug can be obtained from a suitable commercial source. Alternatively, the GPE-related compound or prodrug can be directly synthesized by conventional methods, such as the stepwise solid phase synthesis method of Merrifield et al., J. Amer. Chem. Soc., 85: 2149-2156 (1963), or other appropriate methods known to those of ordinary skill in chemical/biochemical synthesis. Synthesis can also involve the use of commercially available peptide synthesizers such as the Applied Biosystems model 430A.

A compound with little or no immunological effect may be administered over long periods, as long as other side effects prove to be unimportant. We propose that oral doses of a GPE-related compound or prodrug (such as GPE itself) may be given over long periods to, for example, sufferers from chronic CNS disturbances such as multiple sclerosis and the like. In this instance the tripeptide nature of GPE should allow its entry into the circulation by direct absorption from the buccal mucosa from a lozenge placed under the tongue. We have shown that GPE is effective by intraperitoneal administration (in young rats), so it is known to be active by routes other than only injection into the CSF.

If desired, a combination of GPE-related compounds may be administered. In addition, one or more GPE-related compounds may be co-ad mistered with one or more other neuroprotective agents. The other neuroprotective agent(s) will generally be protective, at least in part, of a population of neural cells which is distinct from the population of neural cells which are protected by GPE or other GPE-related compounds. Examples of suitable other neuroprotective agents are insulin-like growth factor-I, insulin-like growth factor-II, transforming growth factor-β1, activin, growth hormone, nerve growth factor, growth hormone binding protein, IGF-binding proteins, basic fibroblast growth factor, acidic fibroblast growth factor, the hst/Kfgk gene product, fibroblast growth factot-3 (FGF-3), FGF-4, FGF-6, keratinocyte growth factor, androgen-induced growth factor, int-2, fibroblast growth factor homologous factor-1 (FHF-1), FHF-2, FHF-3, FHF-4, keratinocyte growth factor 2, glial-activating factor, FGF-10, FGF-16, ciliary neurotrophic factor, brain derived growth factor, neurotrophin 3, neurotrophin 4, bone morphogenic protein 2 (BMP-2), glial-cell line derived neurotrophic factor, activity-dependant neurotrophic factor, cytokine leukemia inhibiting factor, oncostatin M, interleukin, interferon-α, interferon-β, interferon-γ, consensus interferon, TNF-α, clomethiazole; kynurenic acid, met-glu-his-phe-pro-gly-pro (Semax®), tacrolimus, L-threo-1-phenyl-2-decanoylarnino-3-morpholino-1-propanol, andrenocorticotropin-(4-9) analog (ORG 2766), dizolcipine, and selegiline. “Co-administered” or “co-administration” includes not only administration within the same pharmaceutical composition, or administration at the same time, but also administration as part of the same course of treatment for the disease or disorder being treated. Thus, co-administration may, for example, include administration of the GPE-related compound continuously such as by means of an intracerebroventricular shunt, and administration of tablets of another neuroprotective agent orally once daily.

The present invention is further illustrated by the following examples. These examples are offered by way of illustration only and are not intended to limit the invention in any manner. The studies described were approved by the Animal Ethical Committee of the University of Auckland.

### EXAMPLE 1

The objective of this example was to determine the effects of administering GPE on the clinical severity of symptoms in a rat model of experimental autoimmune encephalitis (EAE). EAE was induced by the administration of an emulsion made up of equal volumes of a freshly prepared suspension of Duncan-Hartley strain guinea pig spinal cord in 0.9% saline (1 g/mL) and a suspension of 5 mg M. tuberculosis (H37RV dried and heat killed) in 1 mL of paraffin oil (Difco Freund's incomplete adjuvant).

Male Lewis strain rats weighing 250-300 g were used in this study. Anesthetized rats each received 4 intradermal inoculations consisting of 0.1 mL of emulsion in each hind footpad and 0.05 mL into each posterolateral nuchal region (area on either side of the neck). Rats were left to recover in groups of 2 per cage. Recovering animals had free daily access to pelleted feed and water and were inspected several times daily for symptoms associated with the development of the disease condition.

There are 5 grades of EAE, which are based on clinical symptoms:

Grade 1 Normal except for flaccid tail.

Grade 2 Weakness of both hindlimbs and tail.

Grade 3 Moderate paraparesis or severe ataxia (lack of limb coordination).

Grade 4 Some forelimb weakness, severe paraparesis, episodes of urinary incontinence.

Grade 5 No hindlimb movement, incontinence, impaired respiration.

Sterile Nunc vials were filled with a pre-measured amount of GPE dry powder (150 μg/rat/dose). The vials were stored at −70° C. until they were required for use. At the time of use, 100 μL of sterile 0.9% saline was used to reconstitute the GPE. All syringes and needles were flushed with freshly made 0.1% bovine serum albumin before being loaded with GPE solution. Vehicle animals were given injections of saline only.

Pairs of rats with clear clinical signs of EAE at 12 days post inoculation were intravenously injected with either vehicle or GPE twice daily for 8 days. During this period of treatment, rats were closely observed for physical indications (increased activity, alertness) of a treatment effect. The observer was blinded with respect to the treatment protocol (GPE or vehicle). On completion of the treatment the rats were euthanized.

The method of scoring adopted was cumulative. Rats were individually assessed for the different parameters below and separately scored for each level. All scores were then added together to obtain an overall cumulative score that graded each animal's performance or response to treatment. This was done daily for the duration of the treatment. As such, the cumulative scores reflected each rat's response to the treatment schedule.

Each animal was weighed and its weight recorded.

Each animal was placed on a raised platform and its tail position observed when it walked across from the platform to its litter box over either one of two connecting planks (width 2.8 cm or 3.5 cm). For rats that handled the broader plank confidently, the narrower plank was used:

Does the rat keep its tail raised when walking?—score 0.

Does the rat keep its tail pointing down? If so, the tonicity of the tail was checked. A finger was used to lift the tail from its ventral surface.

If the rat was able to curl its tail around a finger—score 0.5.

If it was unable to do this, then tail very weak but with a little or some muscle tone—score 0.75.

Weak with no tone at all/totally flaccid—score 1.0.

Gait while in the litter box: The gait of individual rats in their cages were observed. The position of each rat's forequarters and hindquarters when they moved around the litter box when the cage lid was removed was noted. Affected rats tended to drag their hindlimbs. The placement of the limbs/paws when at rest were noted.

Gait when walking a plank: Two strips of wood were used. One was somewhat narrower than the other. The broader piece was used for initial assessments and were given the scores according to the following:

Runs across the plank confidently—Score 0.

Runs with hesitation, lacks confidence. Several trials were performed. If the outcome was uncertain, a narrower plank was used.

If the rat still walked hesitantly—Score 0.5.

If the rat was unable to properly place its hind legs—Score 1.0.

Rats that were unable to walk on the plank: The rats which showed a tendency to drag their hind limbs would have previously been detected. Since these rats were also unable to walk on the wooden strip, they were given an additional score of 1.0.

Rats that showed forelimb weakness:

Intermittent forelimb weakness (abnormally positioned paw/limb only when at rest)—score 0.5.

Clearly evident forelimb weakness when attempting to move—score 1.0.

Rats with severe deficits: Rats with severe deficits including impaired breathing, no hind limb movement, and a palpable bladder (suggesting urinary incontinence)—score 1.0

The minimum attainable score was 0 and the maximum score was 5.

Theoretical Example 1: A rat shows the following during testing on the first day: walks the plank hesitantly (0.5), tail is flaccid with some tone (0.75), no other deficiencies (0).

Total score=1.25.

Theoretical Example 2: A rat has a flaccid tail (no tone at all, 1.0), is unable to walk across on the plank (1.0) and also shows some partial limb paralysis (1.0) and forelimb weakness manifested by abnormal forelimb/paw posture while at rest (0.5). Total score=3.5.

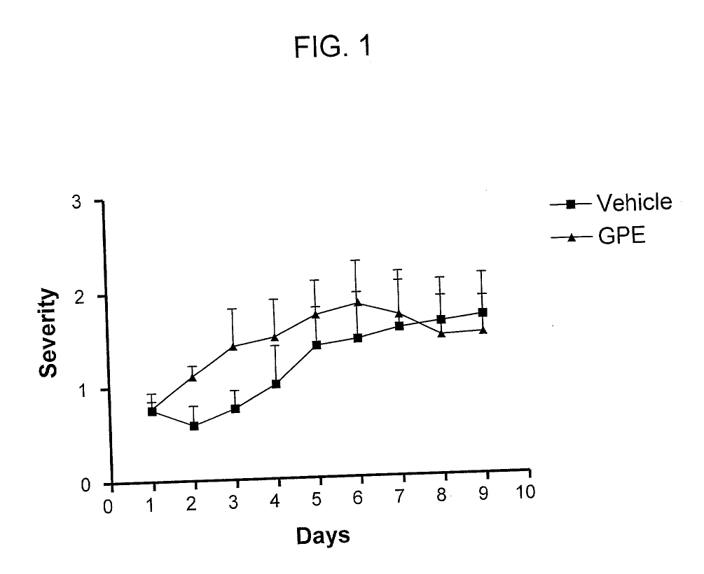

FIG. 1 shows that GPE reduced the clinical severity between days 8 and 9 after inoculation. This indicates that the administration of GPE is effective in treating clinical symptoms.

### EXAMPLE 2

The objective of this example was to determine the effects of administering GPE on the clinical severity of symptoms in EAE, a mouse model of multiple sclerosis (MS). C57BL/6 mice (8-10 weeks old) were injected subcutaneously in one flank with 300 μg of MOG₃₅₋₅₅ peptide, MEVGWYRSPFSRVVHLYRNGK, (Mimotapes Pty Ltd, Clayton, Australia) emulsified in CFA containing 500 μg of M. tuberculosis H37Ra Difco Laboratories, Detroit, USA). They also received 500 ng of pertussis toxin (List Biological Laboratories, CA, USA) in 200 μL PBS intravenously via the tail vein, followed 48 hours later by a second dose. A second injection of MOG₃₅₋₅₅ peptide was given one week later in the opposite flank, without pertusssis toxin injection (Kanwar et al., Immunol. Cell Biol., 78: 641-646 (2000)).

Two treatment protocols were employed, comparing the treatment of early disease and advanced disease. For early disease, reagents were administered at day 35 following MOG₃₅₋₅₅ peptide injection. Anti-MAdCAM-1 monoclonal antibody was administered three times on alternate days and neuroprotectors were given daily for 6 days, where 50% of each neuroprotector was given intraperitoneally and 50% intravenously. In the treatment of advanced disease, therapy was initiated at day 60, with either three or eight anti-MAdCAM-1 monoclonal antibody injections, and neuroprotectors given daily for either 7 or 18 days.

The mice were monitored daily and scored according to the following scale:

| | |
|---|---|
| 0 | no clinical signs of EAE; |
| 1 | limp tail; |
| 2 | partial hind limb paralysis; |
| 3 | complete hind limb paralysis; |
| 4 | complete hind limb and partial fore limb paralysis; |
| 5 | paralysis extending to diaphragm; |
| 6 | hind and fore limb paralysis; |
| 7 | death due to EAE. |

Paralysis extending to the diaphragm was denoted as difficulty in remaining uptight. The mice had to be maintained in a state of hind-limb paralysis, hence care was shown in the provision of dining water, food, and comfort. The daily mean clinical score for each group was the mean disease score of at least five mice. The body weight of animals was measured regularly throughout the experiments. The expanded disability status scale for EAE treated mice was scored using an earlier published scoring system (Villoslada et al., J. Exp. Med., 191: 1799-1806 (2000)).

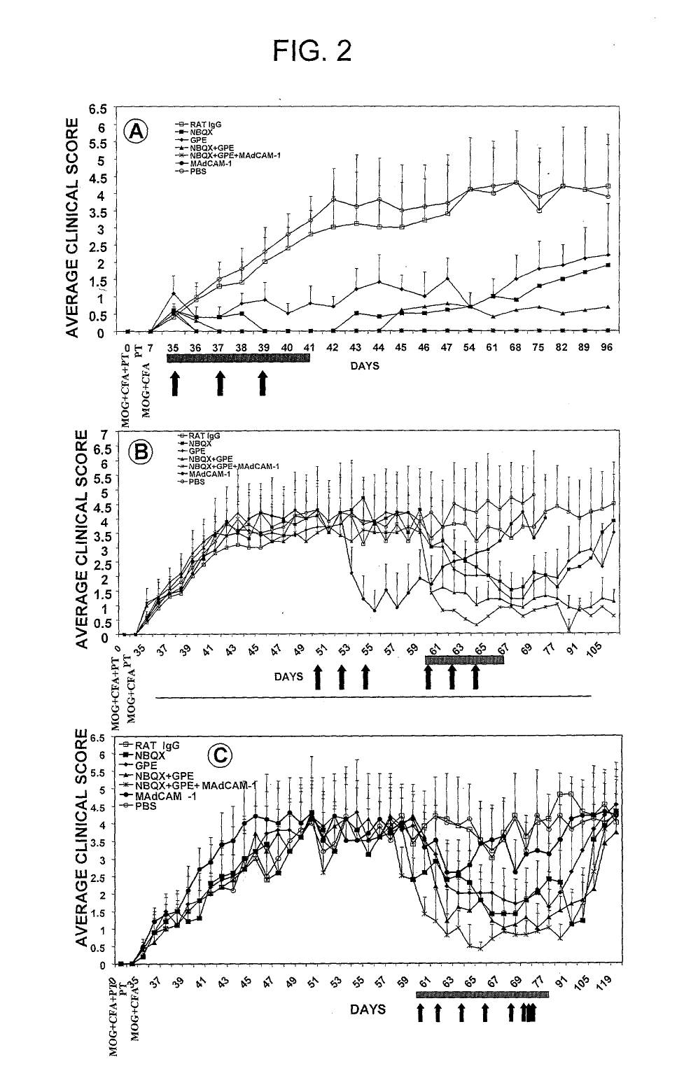

The rat hybridoma MECA-367 (rat IgG2a), which secretes a monoclonal antibody against mouse MAdCAM-1 was either provided by Dr Eugene Butcher, Stanford University, Stanford, Calif. (FIGS. 1A and B), or obtained directly from the American Type Culture Collection, Rockville, Md. (FIG. 1C, and remainder of studies). Rat IgG obtained from Sigma Co., USA, served as a control. Antibodies were administered into the tail vein (50% of mAb) and intraperitoneally (50% of mAb) at 500 μg/rat separately or in combination on alternate days at the time points indicated (FIGS. 2-4). The rat IgG control antibody was also administered at 10 mg/Kg in the same manner. GPE was administered at 30 μg/rat and 1,2,3,4-tetrahydro-6-nitro-2,3-dioxo-benzo[f]quinoxaline-7-sulfonamide (NBQX) at 6 mg/rat on the days indicated, also 50% into the tail vein and 50% intraperitoneally. In the Figures, arrows indicate days of antibody administration and the speckled line indicates days of neuroprotectant administration. Backbones from sacrificed mice were frozen in isopentane at −70° C. Transverse 10 μm sections made through the spinal cord of control and diseased mice, in each case made at the same levels of the cord, were mounted on poly-L-lysine-coated slides, and stained with hematoxylin and eosin.

An antibody to exon-2 of MBP was raised in a rabbit by coupling a mouse MBP exon-2 peptide (H-DSHTRTTHYGSLPQKSQHGRTQDENPVVHFFKNCG-OH) to the carrier protein thyroglobulin. Rabbits were immunized four times with 0.5-1 mg of peptide at 2 to 4 week intervals. The antibody titer was determined at 1:8000 by ELISA. The antibody was used to stain the spinal cord sections at 1:50 dilution.

Mice were perfused through the left cardiac ventricle with ice-cold PBS or glutaraldehyde, and the CNS including brain and spinal cord were prepared for frozen or epoxy sections, respectively. Thin slices were taken at all levels of the CNS including spinal cord (cervical, midthoracic, T11, T12/13, upper lumbar, L6, L7 and sacral) and sacral roots. One micrometer thick epoxy sections taken from different levels of the spinal cord were stained with toluidine blue, and examined by light microscopy by an investigator blinded to the sample identity. Multiple sections from the spinal cord were scored from 0 to 5 for inflammation, deruyelination, remyelination, and axonal necrosis or damage, using a previously published scoring system (Moore et al., Lab. Invest., 51: 416-424 (1984)).

Frozen sections from lumbar spinal cord (10 μm) were acetone-fixed and immunostained for oligodendrocyte content with an antibody against CNPase (Sigma; diluted 1:100); for axonal damage with an antibody against non-phosphorylated neurofilament-H (SM1-32; Sternberger Monoclonals Incorporated, Lutherville, Md., USA; diluted 1:1000), and for GluR2 and NR1 content using mouse mAbs obtained from Zymed Laboratories Inc., Carlton, San Francisco, Calif. Antibody staining was visualized with an avidin:biotinylated enzyme complex (Vector Laboratories, Burlingame, Calif., USA). Sections were viewed under a light microscope, and cells stained with the anti-CNPase and SM1-32 antibodies were counted.

PBS-perfused spinal cords were homogenized in lysis buffer (50 mM Tris pH 7.4, 100 μM EDTA, 0.25 M sucrose, 1% sodium dodecyl sulfate (SDS), 1% Nonidet® P-40 (nonoxynol-9, NP40), 1 μg/mL leupeptin, 1 μg/mL pepstatin A, and 100 μM phenylmethylsulfonylfluoride) at 4° C. using a motor-driven homogenizer (Virtus, Gardiner, N.Y.). Spinal cord lysates from each group of mice were pooled and centrifuged at 10,000×g for 10 minutes at 4° C. to remove tissue debris. Protein concentrations of the supernatants were determined as described (Peterson, Methods Enzymol., 91: 95-119 (1983)), and 100 μg of protein resolved on 10% polyacrylamide SDS-gels under reducing conditions and then electrophoretically transferred to Hybond C Extra nitrocellulose membranes (Amersham Life Science, England). The membranes were blocked with 3% bovine serum albumin in TBS-T (20 mM Tris, 137 mM NaCl, pH 7.6, containing 0.1% Tween 20) for 2 hours at room temperature. Immunodetection was accomplished by incubation overnight at 4° C. with SM1-32 (1:500 dilution), anti-CNPase (1:100 dilution) antibody, or anti-GluR2 and NR1 mAbs (1: 200 dilution). The membranes were washed three times with TBS-T and incubated with horseradish peroxidase-conjugated anti-mouse IgG (Sigma) diluted 1:5000 in TBS-T. Immunoreactivity was detected by Enhanced Chemiluminescence (Amersham International plc. England) and autoradiography.

Cells undergoing apoptosis were identified using the in situ TUNEL assay. Five micrometer thick serial sections of brain and spinal cord were prepared to detect apoptosis by TUNEL (i.e., terminal deoxynucleotidyl transferase-mediated deoxyuridine triphosphate-digoxigenin nick end labeling) staining using the In Situ Apoptosis Detection Kit from Boehringer Mannheim (Germany). Briefly, frozen sections were fixed in 4% paraformaldehyde, permeabilized in 0.1% Triton X-100, incubated with 20 μL TUNEL reagent for 60 minutes at 37° C., and then examined by fluorescence microscopy. Adjacent sections were counterstained with hematoxylin and mounted onto poly-L-lysine coated slides to allow the total number of nucleated cells to be counted. The percentage of apoptotic cells was assessed in ten randomly selected fields viewed at 40×magnification. The apoptotic index (AI) was calculated as AI=number of apoptotic (TUNEL-positive) cells×100/total number of nucleated cells.

The first signs of clinical EAE ensued between days 28 to 35, depending on the particular batch of MOG₃₅₋₅₅ peptide autoantigen, leading to chronic sustained paralysis of the hind limbs eight days later (FIG. 2). The prolonged persistence of clinical symptoms at the same level for several months is characteristic of MOG-induced EAE in the C57BL/6 strain of mice (Mendel et al., Eur. J. Immunol., 25:1951-1959 (1995)). Three iv and ip injections of anti-MAdCAM-1 mAb, given on days 35,36, and 37 following injection of autoantigen completely prevented the induction of EAE such that no overt clinical symptoms could be observed for 60 days after suspension of antibody treatment (FIG. 2A).

GPE given daily from day 35 for 6 days suppressed EAE, up to 27 days after suspension of treatment (disease score 0.4 to 2.2 compared to 2.6 to 5.9 for controls). However, thereafter disease severity gradually increased. NBQX given daily for 6 days also suppressed EAE, but to a greater extent than achieved with GPE, however as with GPE the severity of disease gradually increased 27 days following suspension of treatment. In contrast, combined treatment with GPE and NBQX led to sustained suppression of disease symptoms, at least for the 55 days the animals were monitored following suspension of treatment. The triple combination of anti-MAdCAM-1 mAb, NBQX and GPE, as with anti-MAdCAM-1 mAb alone completely prevented the development of disease in all mice.

As described previously, anti-CAM therapy to block inflammation is only preventative when administered early, prior to establishment of significant nerve damage. Thus, three injections of anti-MAdCAM-1 mAb at high dose starting on day 51 caused initial remission of disease, but this was quickly followed by a gradual and complete relapse after suspension of treatment (FIG. 2B). NBQX and GPE administered for 7 days as monotherapies only caused temporary remission, and by day 105 the disease had almost returned to control levels of crippling paralysis. In contrast, the combination of NBQX with GPE appeared to be synergistic as the disease was suppressed (disease score reduced from 4.5 to 1) for 40 days following suspension of treatment (FIG. 2B). This indicates that administering a combination of GPE and NBQX delays disease progression. It makes no sense to inhibit demyelinating inflammation without attempting repair of the CNS; or achieving repair without damage limitation.

For the first time we have combined both approaches to successfully treat advanced disease. Remarkably, simultaneous administration of anti-MAdCAM-1 mAb, NBQX and GPE delivered sustained protection (disease score reduced from 5 to <1) against disease progression.

The various treatment regimes were administered for 16 days in order to determine whether prolonged treatment was non-toxic (FIG. 2C). In this experiment, similar results were obtained as in FIG. 2B. Thus the effectiveness of the treatments was in the order NBQX+GPE+anti-MAdCAM-1>NBQX+GPE>NBQX>GPE; whereas anti-MAdCAM-1 monotherapy only weakly attenuated disease severity. However, the disease relapsed in all cases following suspension of treatment, suggesting that therapy must be maintained if it is to be consistently effective.

Two mice died for unknown reasons in the NBQX+GPE combination, but not in the NBQX+GPE+anti-MAdCAM-1 combination. Since no mice from the triple combination, or the earlier experiment (FIG. 2B) had died, the combinational treatment protocols appear to be safe and effective at the doses employed.

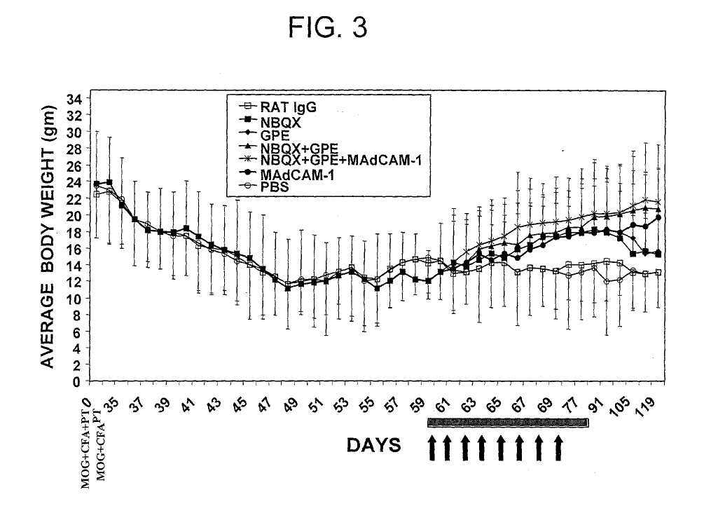

Weight loss was found to correlate with disease severity. At disease onset the body weight of mice rapidly decreased, such that mice had lost 50% of their average body weight within 2 weeks of disease progression (FIG. 3). Mice experienced a weight gain following treatment that correlated with the efficacy of the treatment regime (i.e. NBQX+GPE+anti-MAdCAM-1>NBQX+GPE>NBQX, GPE, or MAdCAM-1 monotherapies), where the weight of mice receiving the triple treatment returned almost to normal.

The disability scores for the various treatment groups correlated well with the clinical scores of paralysis. Diseased mice displayed discernible impairments in spontaneous mobility, tone, motor function (grip), sensory function, and shiny hair/skin firmness. The total clinical scores inversely correlated with the efficacy of the particular treatment regime (i.e. NBQX+GPE+anti-MAdCAM-1>NBQX+GPE>NBQX or GPE or anti-MAdCAM-1 mAb). For example, the total scores at day 70 were 3 for NBQX+GPE+anti-MAdCAM-1, 10 for NBQX+GPE, 10 for GPE, 30 for NBQX, 19 for anti-MAdCAM-1 mAb, and 35 for rat IgG treated control mice).

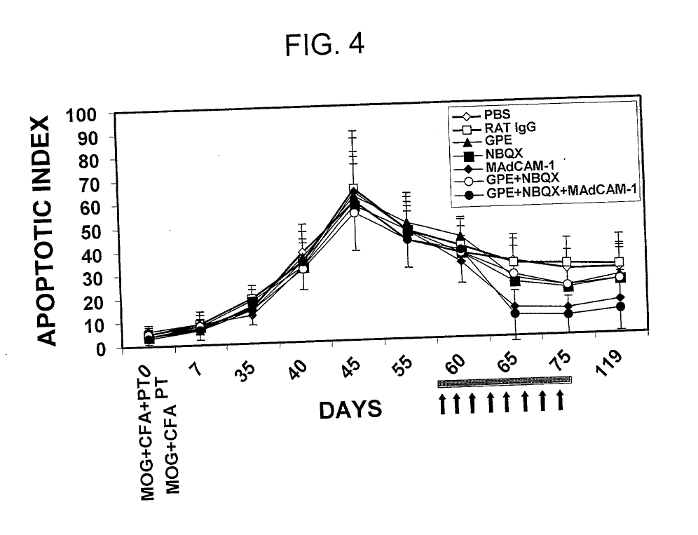

Neuropathological evaluation confirmed the observed clinical protection achieved with the various treatment regimes. Spinal cord sections stained by TUNEL analysis revealed extensive apoptosis which peaked at the height of disease severity (day 49), and then subsequently declined slightly to reach a plateau that was maintained for the duration of the experiment (FIG. 4). Apoptotic cells were not identified but it is probable that infiltrating T cells, macrophages, and resident microglia and oligodendrocytes are all represented. Infiltrating T cells in particular appear to undergo apoptosis, as the body tries to clear autoimmune inflammation.

Sections taken from NBQX+GPE+anti-MAdCAM-1, and anti-MAdCAM-1 treated mice had substantial reductions (60%) in the numbers of apoptotic cells compared to mice that had been mock treated with either PBS or rat IgG. In contrast, sections taken from NBQX+GPE, NBQX, and GPE treated mice showed lesser reductions (˜30%) in the number of apoptotic cells.

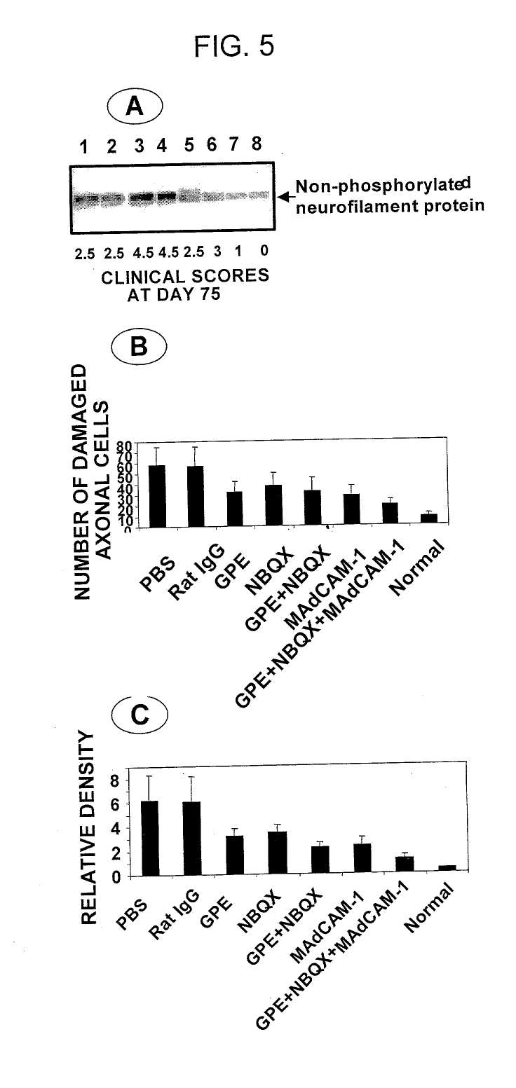

Axonal damage is a critical feature of multiple sclerosis lesions. Dephosphorylated heavy chain neurofilament-H is a quantitative molecular marker of demyelinated and dystrophic axons, and is employed to assess axonal damage and disease severity. FIG. 5A is a Western blot analysis of dephosphorylated neurofilament H in spinal cord homogenates. PBS-perfused spinal cords were homogenized, and proteins Western blotted for dephosphorylated neurofilament H with the SM1-32 mAb. Immunoreactivity was detected by enhanced chemiluminescence and autoradiography. Homogenates were derived from mice treated with GPE ([ane 1), NBQX (lane 2), PBS (lane 3), rat IgG (lane 4), GPE+NBQX (lane 5), anti-MAdCAM-1 mAb (lane 6), NBQX+GPE+anti-MAdCAM-1 mAb (lane 7), and from untreated normal mice (lane 8). Clinical scores at day 75 are indicated beneath the panel. FIG. 5B shows the numbers of damaged axonal cells (cells staining for dephosphorylated neurofilament H). Data represent the means±s.e.m. FIG. 5C is a densitometric analysis of Western blots of total spinal cord homogenate of 5 representative mice per group. Data represent the means±s.e.m. Using both immunohistochemistry (FIG. 5B) and Western blot analysis (FIGS. 5A and 5C), it was revealed that the spinal cords of mock-treated EAE mice (disease score 4.5) displayed a large increase of abnormally dephosphorylated neurofilament-H, whereas normal undiseased mice had almost undetectable levels (FIGS. 5A and 5B). In accord these spinal cords also contained increased numbers of damaged axonal cells (FIG. 5B). Once again, the levels of dephosphorylated neurofilament-H, and numbers of damaged axonal cells correlated with the efficacy of the particular treatment regime. For example the average relative densities of neurofilament-H in spinal cord homogenates resolved by Western blot analysis were 1.2 for NBQX+GPE+anti-MAdCAM-1, 2.2 for NBQX+GPE, 2.3 for anti-MAdCAM-1, 3.2 for GPE, and 3.5 for NBQX, compared to 6.1 for either rat IgG or PBS treated control mice). This indicates that the triple combination was very effective in reducing axonal damage almost to background levels.

GPE and NBQX administered individually almost halved the amount of axonal damage, and in combination further reduced damage by 30%, indicating the involvement of glutamate excitotoxicity. Surprisingly, anti-MAdCAM-1 mAb treatment was almost as effective as the combination of GPE+NBQX, which correlated with its ability to reduce the apoptotic index (FIG. 4).

These results clearly indicate that the anti-inflammatory reagents and neuroprotectors used act in concert to reduce the degree of axonal damage, as reflected in the attenuation of clinical symptoms of disease.

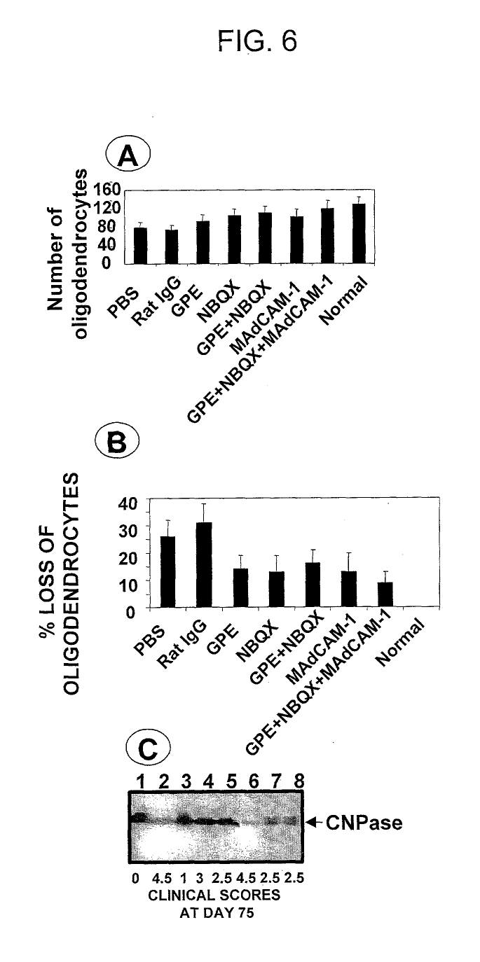

To evaluate the effects of the different treatment regimes on the loss of oligodendrocytes, a key cellular target in demyelinating diseases of the CNS, oligodendrocytes were enumerated by immunohistochemical staining of spinal cord sections and the number of oligodendrocytes within the dorsal columns of the transverse sections were counted (FIG. 6A: each bar represents the average number of oligodendrocytes from two representative mice, with 5-10 sections analyzed pet mouse), and reductions in oligodendtocyte numbers were calculated and expressed as percentage loss of oligodendrocytes per dorsal section (FIG. 6B: data ate expressed as the percentage±s.e.m. of cells lost per dorsal column). The loss of oligodendrocytes correlated with the efficacy of the different treatment regimes, such that the loss was 9% for NBQX+GPE+anti-MAdCAM-1, 16% for NBQX+GPE, 13% for anti-MAdCAM-1, 14% for GPE, 13% for NBQX, 31% for rat IgG, and 26% for PBS treated control mice. FIG. 6C is a Western blot analysis of CNPase in spinal cord homogenates. PBS-perfused spinal cords were homogenized, and proteins Western blotted with an anti-CNPase mAb. Immunoreactivity was detected by enhanced chemiluminescence and autoradiography. Homogenates were derived from normal undiseased mice (lane 1), and from EAE mice treated with PBS (lane 2), NBQX+GPE+anti-MAdCAM-1 mAb (lane 3), anti-MAdCAM-1 mAb (lane 4), GPE+NBQX (lane 5), rat IgG (lane 6), NBQX (lane 7), and GPE (lane 8). Clinical scores at day 75 are indicated beneath the panel.

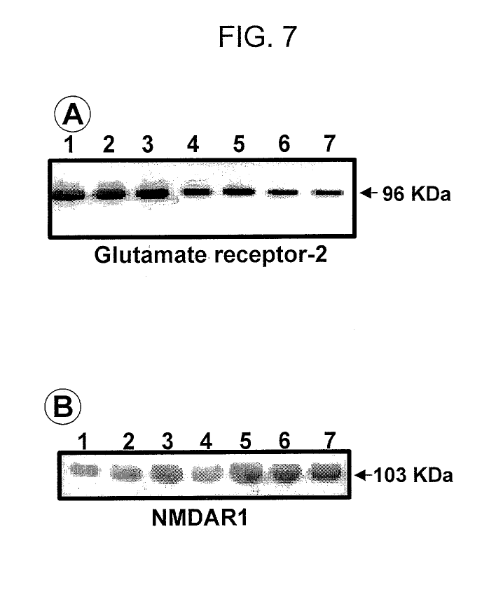

Levels of the AMPA receptor subunits GluR1 and 2 have been shown to increase in the rat spinal cord after inflammation, suggesting their upregulation is an indicator of disease (Zhou et al., Brain Res. Mol. Brain. Res., 88: 186-193 (2001)). Thus levels of GluR2 and the NMDA receptor subunit NR1 were determined in the current investigation. FIG. 7A is a Western blot analysis of GluR2 subunit expression in spinal cord homogenates. PBS-perfused spinal cords were homogenized, and proteins Western blotted with an anti-GluR2 mAb. Homogenates were derived from mice treated with rat IgG (lane 1), GPE (lane 2), anti-MAdCAM-1 mAb (lane 3), NBQX+GPE+anti-MAdCAM-1 mAb (lane 4), GPE+NBQX (lane 5), NBQX (lane 6), and from normal mice (lane 7). FIG. 7B is a Western blot analysis of NMDAR1 subunit expression in spinal cord homogenates. PBS-perfused spinal cords were homogenized, and proteins Western blotted with an anti-NMDAR1 mAb. Homogenates were derived from normal undiseased mice (lane 1), and mice treated with treated with NBQX (lane 2), NBQX+GPE+anti-MAdCAM-1 mAb (lane 3), GPE+NBQX (lane 4), GPE (lane 5), anti-MAdCAM-1 mAb (lane 6), and PBS (lane 7). Immunoreactivity was detected by enhanced chemiluminescence and autoradiography. As shown by both Western blot analysis of spinal cord homogenates (FIGS. 7A and 7B) there were marked increases in GluR2 and NR1 subunit expression in the spinal cords of mock-treated EAE mice. Treatments that included NBQX led to a marked down-regulation of GluR2 expression, whereas GPE and anti-MAdCAM-1 mAb reagents had negligible effect. Thus, NBQX either directly or indirectly downregulates the AMPA receptor following binding. Surprisingly, NBQX also caused down-regulation of the NMDA receptor, whereas anti-MAdCAM-1 mAb and GPE were not effective. This is in accord with a study showing that drug regimes targeting one ionotropic glutamate receptor subtype may indirectly affect other subtypes (Healey et al., Synapse, 38: 294-304 (2000)).

GPE is effective in protecting white matter against degeneration including the degeneration of axons and the death of oligodendtocytes. GPE and other GPE-related compounds therefore have specific application in treating disorders and disease which affect white matter, axons and oligodendtocytes, particularly multiple sclerosis and periventicular leucomalacia.

Although the present invention is defined broadly above, it will be appreciated by those skilled that it is not limited thereto but includes embodiments of which the description provides examples. Finally, it will be appreciated that various alterations and modifications may be made to the foregoing without departing from the scope of this invention as claimed.

We claim:

1. A method for protecting white matter, axons, and oligodendrocytes of a mammal, against degeneration and death resulting from multiple sclerosis or periventricular leucomalacia comprising increasing the effective concentration of a GPE-related compound in the central nervous system of the mammal.

2. The method as claimed in claim 1 wherein the mammal is human.

3. The method of claim 1 where the GPE-related compound is GPE.

4. The method of claim 1 where the GPE-related compound is a GPE analog.

5. The method of claim 4 where the GPE analog is selected from the group consisting of GP, PE, GP (E-amide), GPE stearate, GP(D-E), GPT, GEP, EGP, and EPG.

6. The method of claim 1 where the mammal is a human.

7. The method of claim 1 comprising administration to the mammal of an effective amount of a GPE-related compound, a prodrug thereof, or an implant containing cells that express the GPE-telated compound or prodrug.

8. The method of claim 7 where the GPE-related compound, prodrug, or implant is administered directly to the cerebral ventricle of the mammal.

9. The method of claim 7 where the GPE-related compound, prodrug, or implant is administered peripherally to the mammal.

10. The method of claim 7 where the GPE-related compound is administered centrally in an amount from about 0.1 μg/Kg/day to about 400 μg/Kg/day.

11. The method of claim 10 where the GPE-related compound is administered centrally in an amount from about 0.5 μg/Kg/day to about 100 μg/Kg/day.

12. The method of claim 11 where the GPE-related compound is administered centrally in an amount from about 1 μg/Kg/day to about 25 μg/Kg/day.

13. The method of claim 7 where the GPE-related compound, prodrug, or implant is administered in combination with artificial cerebrospinal fluid.

14. The method of claim 7 where the GPE-related compound is co-administered with an other neuroprotective agent.

15. The method of claim 14 where the other neuroprotective agent is selected from insulin-like growth factor-I, insulin-like growth factor-II, transforming growth factor-β1, activin, growth hormone, nerve growth factor, growth hormone binding protein, IGF-binding proteins, basic fibroblast growth factor, acidic fibroblast growth factor, the hst/Kfgk gene product, fibroblast growth factor-3 (FGF-3), FGF-4, FGF-6, keratinocyte growth factor, androgen-induced growth factor, int-2, fibroblast growth factor homologous factor-1 (FHF-1), FHF-2, FHF-3, FHF-4, keratinocyte growth factor 2, glial-activating factor, FGF-10, FGF-16, ciliary neurotrophic factor, brain derived growth factor, neurotrophin 3, neurotrophin 4, bone morphogenic protein 2 (BMP-2), glial-cell line derived neurotrophic factor, activity-dependant neurotrophic factor, cytokine leukemia inhibiting factor, oncostatin M, interleukin, interferon-α, interferon-β, interferon-γ, consensus interferon, TNF-α, clotmethiazole; kynurenic acid, met-glu-his-phe-pro-gly-pro (Semax®), tacrolimus, L-threo-1-phenyl-2-decanoylatino-3-morpholino-1-propanol, andrenocorticotropin-(4-9) analog (ORG 2766), dizolcipine, and selegilne.

16. The method of claim 7 where the GPE-related compound, prodrug, or implant is administered subsequent to the onset of multiple sclerosis but prior to degeneration or death of white matter, axons, or oligodendrocytes.

17. The method of claim 7 where the GPE-related compound, prodrug, or implant is administered subsequent to the onset of periventricular leucomalacia but prior to degeneration or death of white matter, axons, or oligodendrocytes.

18. The method of claim 1 where the GPE-related compound or prodrug is administered a pharmaceutical composition.
